(19) United States
(12) Patent Application Publication
(10) Pub. No.: US 2018/0360342 A1
(43) Pub. Date: Dec. 20, 2018

(54) RENAL ABLATION AND VISUALIZATION SYSTEM AND METHOD WITH COMPOSITE ANATOMICAL DISPLAY IMAGE

(71) Applicant: BIOSENSE WEBSTER (ISRAEL) LTD., YOKNEM (IL)

(72) Inventors: Kristine Fuimaono, Costa Mesa, CA (US); Debby Highsmith, Laguna Niguel, CA (US)

(21) Appl. No.: 15/625,820

(22) Filed: Jun. 16, 2017

(51) Int. Cl. A61B 5/055; A61B 34/10; A61B 34/20; A61B 5/00; A61B 18/14; A61B 90/00

(52) U.S. Cl. CPC A61B 5/055; A61B 34/10; A61B 34/20; A61B 2017/00053; A61B 18/1492; A61B 90/37; A61B 5/414

(57) ABSTRACT

A method and device that provide improved visualization of soft tissue, such as renal arteries, renal veins and lymph nodes in guiding catheter placement and positioning in the renal region or vasculature. The method and device enable visualization of an electrophysiology catheter application in the renal region which provides for improved imaging of renal structures, including renal arteries, along with one or more adjacent anatomical structures, including renal veins, lymph nodes, other adjacent organs and and/or other adjacent soft tissues that may adversely impact the formation of a lesion during a catheter ablation procedure in or around a renal artery.

### FIELD OF INVENTION

Aspects of embodiments of the present invention relate to invasive medical devices and associated systems, and related methods, capable of ablation and visualization of catheter and anatomical structures.

### BACKGROUND OF INVENTION

Catheterization is used in diagnostic and therapeutic procedures. For example, a cardiac catheter is used for mapping and ablation in the heart to treat a variety of cardiac ailments, including cardiac arrhythmias, such as atrial flutter and atrial fibrillation which persist as common and dangerous medical ailments, especially in the aging population. Diagnosis and treatment of cardiac arrhythmias include mapping the electrical properties of heart tissue, especially the endocardium and the heart volume, and selectively ablating cardiac tissue by application of energy. Such ablation can cease or modify the propagation of unwanted electrical signals from one portion of the heart to another. The ablation process destroys the unwanted electrical pathways by formation of non-conducting lesions. Various energy delivery modalities have been disclosed for forming lesions, and include use of microwave, laser and more commonly, radiofrequency energies to create conduction blocks along the cardiac tissue wall. In a two-step procedure—mapping followed by ablation—electrical activity at points within the heart is typically sensed and measured by advancing a catheter containing one or more electrical sensors (or electrodes) into the heart, and acquiring data at a multiplicity of points. These data are then utilized to select the endocardial target areas at which ablation is to be performed.

The term “radiofrequency” (RF) is commonly used to refer to an alternating current that flows through a conductor. In the case of ablation, RF current flows through biological tissue that contains free ions. The extra cellular fluid present in the tissue provides the electrical conductivity. The tissue conductivity can be represented by tissue impedance. In general, low impedance represents high conductivity and high impedance represents low conductivity.

The application of RF current biological tissue causes heating of tissue. The higher the RF current density in the biological tissue (current per unit area), the higher the resulting temperature. The tissue stops reacting to electrical stimulation when heated above a threshold over a short period.

Another catheter-based ablation procedure is renal denervation (RDN). It is a minimally invasive, endovascular catheter based procedure using radiofrequency ablation aimed at treating medical conditions and diseases, including, for example, hypertension. The sympathetic system fuels the release of certain hormones that affect and control blood pressure. In hypertension, the continued release of low-dose amounts of these hormones can increase blood pressure. Hypertension can be controlled by diet, exercise and drugs. However, resistant hypertension (commonly defined as blood pressure that remains above goal in spite of concurrent use of three antihypertensive agents of different classes) requires more aggressive treatments, including surgery. Resistant hypertension is a common clinical problem faced by both primary care clinicians and specialists. As older age and obesity are two of the strongest risk factors for uncontrolled hypertension, the incidence of resistant hypertension will likely increase as the population becomes more elderly and heavier.

It has been established that severing the renal nerves improves blood pressure. However, this procedure involves surgery and all its attendant risks, and often resulted in global sympathetic denervation below the chest. Being able to de-nervate, or silence, only the renal nerves through a catheter-based system is a crucial development. A small catheter is placed in the femoral artery and access to the nerves is gained through the renal artery. The nerves are woven and embedded in the casings or layers around the renal arteries. By passing an energy source into the renal artery and transmitting a low-dose energy, radiofrequency ablation, through the catheter, inbound and exiting renal sympathetic nerves are exposed to RF current densities. The extent of heating is proportional to the RF power (current density) output. At low current densities, the tissue is heated slowly and contracts because of fluid loss. With the nerves impaired or “denerved” at selected locations along their lengths, sympathetic afferent and efferent activity is interrupted or reduced with beneficial effects, such as a reduction in blood pressure.

Catheter-based renal denervation is typically performed under fluoroscopy where X-ray imaging provides 2-D visualization of the catheter in the renal vasculature, including the renal artery where ablation is performed. Different parts of the body absorb the x-rays in varying degrees. Dense bone absorbs much of the radiation while soft tissue, such as muscle, fat and organs, allow more of the x-rays to pass through them. As a result, bones appear white on the x-ray and soft tissue is displayed in shades of gray and air appears black. Visualization of anatomical structures and catheter placement is therefore limited. Fluoroscopy may not provide adequate visualization of soft tissue such as renal arteries, renal veins and lymph nodes.

Computed tomography (CT or CAT scan) can produce images of soft tissue, internal organs, bones and blood vessels in greater detail than traditional X-rays. Cross sectional-images generated during a CT scan can be reformatted in multiple planes and can even generate three-dimensional images which can be viewed on a computer monitor, printed on film or transferred to a CD or DVD. Similarly, magnetic resonance imaging (MRI) uses magnetic field and pulses of radio waves to produce images of organs and structures the body. In many cases, MRI can provide different visualization of organs and structures compared to X-ray, ultrasound and CT scan. Contrast agents may also be used during the MRI scan to show certain structures more clearly. The images are digitized and can be saved and store on a computer or reviewed remotely. Improvements in magnetic resonance hardware, scanning protocols and 3D volumetric reconstruction software have enabled three-dimensional imaging.

Conventional ultrasound provides an image in two dimensions, using sound waves that have been transmitted through the body and bounced off internal organs and structures. The collected sound waves are processed by a computer to create an image. The two-dimensional images are displayed in thin, flat sections. In three dimensional scanning, instead of the sound waves being transmitted at one angle, they are transmitted at different angles with the returning echoes being processed by a sophisticated computer program resulting in a reconstructed three-dimensional volume image of internal organs and structures, in much the same way as a CT scan machine constructs a CT scan image from multiple x-rays. The resulting images provide depth and shadows, and thus better visualization of details.

More recently, fluoroscopy or computed tomography may be used to complement anatomic mapping in order to produce a visual reconstruction of cardiac chambers. An example is described in commonly assigned application Ser. No. 13/295,594, the entire content of which is herein incorporated by reference. A position processor accurately relates the position of the tip of an ablation catheter to target areas using the reconstruction, in order to assure contact between an ablation electrode at the catheter tip and the endocardial surface.

U.S. Publication No. US 2007/0049817 A1, the entire content of which is herein incorporated by reference, discloses systems and methods for registering maps with images, involving segmentation of three-dimensional images and registration of images with an electro-anatomical map using physiological or functional information in the maps and the images, rather than using only location information. A typical application of the invention involves registration of an electro-anatomical map of the heart with a preacquired or real-time three-dimensional image. Features such as scar tissue in the heart, which typically exhibits lower voltage than healthy tissue in the electro-anatomical map, can be localized and accurately delineated on the three-dimensional image and map.

U.S. Pat. No. 7,831,076 discloses methods and apparatuses for a 3-D model of a structure being imaged, e.g., an electro-anatomical map, to be co-displayed and visually marked, to indicate progress of data acquisition, during acquisition of ultrasound data in a medical imaging procedure. The plane of intersection of successive two-dimensional images is marked as a line or colored region on the three-dimensional model. This display enables the operator to determine regions where sufficient data have been captured, and guides the operator to areas where additional data collection is still needed. Various color schemes are used to indicate the relative sufficiency of data collected.

U.S. Pat. No. 9,078,567 discloses methods and apparatuses for visually supporting an electrophysiology catheter application in the heart, whereby electro-anatomical 3D mapping data of an area of the heart to be treated which are provided during performance of the catheter application are visualized. Before the catheter application is carried out, 3D image data of the area to be treated are recorded by means of a tomographical 3D imaging method, a 3D surface profile of objects in the area to be treated is extracted from the 3D image data by segmentation and the electro-anatomical 3D mapping data provided and the 3D images representing the 3D surface profile are associated with each other in the correct position and dimension relative each other and e.g. visualized in an superimposed manner during the catheter application. The methods and the corresponding devices allow for an improved orientation of the user who carries out an electrophysiology catheter application in the heart.

### SUMMARY OF THE INVENTION

The methods and systems of present invention recognize that renal veins tend to track closely to renal arteries and that lesion geometry changes when there is a vein in the field of RF energy. For example, the lesions tend to be smaller resulting in less effective denervation. Other adjacent anatomical structures or anatomical structures in close or immediate proximity (used interchangeably herein) to renal arteries, such as lymph nodes, have also been observed to adversely impact lesion geometry.

It is an object of some embodiments of the present invention to specify a method and a device that provide improved visualization of soft tissue, such as renal arteries, renal veins and lymph nodes in guiding catheter placement and positioning in the renal region or vasculature. In some embodiments, the method and device enables visualization of an electrophysiology catheter application in the renal region which provides for, for example, improved imaging of renal structures, including renal arteries, along with one or more adjacent anatomical structures, including renal veins, lymph nodes, other adjacent organs and and/or other adjacent soft tissues that may adversely impact the formation of a lesion during a catheter ablation procedure in or around a renal artery.

In the present method of at least one embodiment, for visually supporting an electrophysiology catheter application in the renal region, particularly a catheter renal ablation, 3-D image data of the area to be treated are provided by way of fluoroscopy or a tomographical 3-D imaging method before or concurrently with a 3-D mapping procedure providing anatomical 3-D mapping data. From the 3D image data, a 3D surface profile of objects, particularly a renal artery and one or more adjacent anatomical structures, in the area to be treated is extracted by segmentation. The 3D image data representing the 3D surface profile, called selected 3D image data in the text that follows, are associated with the anatomical 3D mapping data provided during the performance of the catheter application in the correct position and dimension. The 3D mapping data and at least the selected 3D image data are then visualized superimposed on one another in the correct position and dimension in a visual representation during the performance of the catheter application, such that adjacent anatomical structures, including, renal veins, lymph nodes, other adjacent organs and/or other adjacent soft tissues, are visualized along with visualization of the renal artery and the ablation catheter.

Due to this superposition of the 3D surface profile, by which the morphology of the renal area to be treated or being treated is reproduced in good quality, with the anatomical 3D mapping data recorded during the performance of the catheter application, a better orientation and more accurate details are conveyed to the operator of the catheter during the performance of the catheter application so that the operator may selectively position the renal catheter in the renal artery, including positioning ablation electrodes of the renal catheter in regions of the renal artery that are more remote from, or are devoid of, adjacent anatomical structures, including, for example, renal veins, lymph nodes, organs and other soft tissues. The superimposed imaging can take place, for example, on a monitor in the control room or in the operating room itself. On the monitor, a cardiologist then recognizes these anatomical structures and is able to intelligently position ablation electrodes of the renal catheter for improving lesion formation, including better lesion quality and size.

For recording the 3D image data, methods of X-ray computer tomography, of magnetic resonance tomography or of 2D or 3D ultrasonic imaging can be used, for example. Combinations of these imaging methods are also possible.

Different techniques can be used for segmenting the 3D image data recorded. Thus, the three-dimensional surface profile of the objects contained in the 3D image data, particularly of the vessels and/or one or more adjacent anatomical structures, can be produced, for example, by segmenting all 2D layers obtained with the imaging method. Apart from this layered segmentation, a 3D segmentation of one or more anatomical structures is also possible. Suitable segmentation techniques are known to the expert in the field of image processing of medical image data.

Correlating the anatomical 3D mapping data with the selected 3D image data in the correct dimension and position can be done by way of different techniques. One possibility resides in registration between the respective data by visually matching the 3D surface profile with the representation of the anatomical 3D mapping data. Furthermore, artificial markers or natural distinct points can be used which can be recognized in both records. Apart from the area to be treated, a neighboring area can also be used for the registration if it is contained in the existing data. Furthermore, it is possible to place the center on data in the environment of the tissue to be removed, also called the target tissue in the text which follows, or of the catheter point during the performance of the registration.

In advantageous embodiments of the method and of the system, the registration takes place in a first stage in which only a relatively small portion of the anatomical 3D mapping data is present, with the aid of artificial markers or of distinct points, and in one or more subsequent stages in which a greater number of anatomical 3D mapping data is already present, by surface matching. In this manner, the registration is improved with the increasing number of anatomical 3D mapping data during the catheter application.

During the superimposition of the anatomical 3D mapping data on the 3D image data, these 3D image data can be represented by way of a volume rendering technique. In a further embodiment, the 3D surface profile is represented by a polygonal grid as is known from the field of computer graphics. The superimposition can be performed with adjustable transparency and adjustable blending factor. It is also possible to calculate and display an endoscopic perspective. Since the anatomical 3D mapping data also contains the respective instantaneous position of the catheter point, it is also possible to visualize only the position of the catheter in real-time in the representation of the 3D image data from time to time without displaying the remaining 3D mapping data.

Furthermore, the distance of the catheter to any picture elements of the 3D image data can be calculated due to the registration between the 3D mapping data and the 3D image data. This is made possible by an advantageous embodiment of the present method in which the catheter point is displayed colored in the visualization, the color changing in dependence on the distance from predeterminable picture elements, particularly the position of the target tissue.

The present system of at least one embodiment for performing the method of at least one embodiment includes one or more input interfaces for the anatomical 3D mapping data and the 3D image data recorded by means of an imaging tomographic method. The device exhibits a segmentation module for segmenting the 3D image data in order to extract a 3D surface profile of objects contained inside the volume recorded by way of the 3D image data. This segmentation module is connected to a registration module which is constructed for correlation with the correct position and dimension of the anatomical 3D mapping data and the 3D image data, representing the 3D surface profile. This registration module, in turn, is connected to a visualization module which superimposes the 3D mapping data and at least the 3D image data representing the 3D surface profile on one another in the correct position with the correct dimension, for visualization by way of a display device, particularly a monitor or projector.

In some embodiments, the methods and devices of the present invention further enable one or more selected anatomical structures to be highlighted, demarcated or “tagged” either manually by the cardiologist, or automatically by the system. For example, one or more anatomical structures targeted for ablation may be tagged. For example, one or more anatomical structures to be avoided in placement of ablation catheter and/or its ablation electrodes may be tagged. Such one or more tagged anatomical structures may be used in the image correlation and/or registration process. Such one or more tagged anatomical structures may be displayed in an enhanced manner for recognition and consideration by the cardiologist as a region in which to place or positioned ablation electrodes, or alternatively, as a region to avoid placement or positioning ablation electrodes.

Furthermore, methods of ablating a renal artery in accordance with some embodiments of the present invention include blocking blood flow in one or more adjacent renal veins during ablation in a renal artery performed during a renal denervation procedure. Blocking blood flow in the renal vein reduces or eliminates cooling effects produced by flowing blood in the renal vein so that lesions can form in an unrestricted manner. Blocking blood flow in the renal vein can be accomplished by, for example, but not limited to introducing a second catheter in the renal vein, and inflating the balloon to temporarily restrict blood flow in the renal vein in a region adjacent the ablation region of the renal artery.

### BRIEF DESCRIPTION OF THE DRAWINGS

These and other features and advantages of the present invention will be better understood by reference to the following detailed description when considered in conjunction with the accompanying drawings wherein:

FIG. 1 is a schematic, pictorial illustration of a catheter-based renal ablation and composite anatomical imaging system, in accordance with an embodiment of the present invention.

FIG. 2A is a schematic view of a renal artery with a catheter extending therethrough.

FIG. 2B is a cross-end sectional view of the renal artery and the catheter of FIG. 2A, taken along line B-B.

FIG. 3 is a side view of a renal ablation catheter of the present invention, in accordance with one embodiment.

FIG. 4 is a schematic block diagram of a portion of the catheter-based renal ablation system of FIG. 1.

FIG. 5 is a schematic block diagram of circuitry used in a catheter-based renal ablation and composite imaging system, in accordance with one embodiment.

FIG. 6 is a flow chart illustrating a method for generating a composite image from a 3-D image data and a 2-D fluoroscopic image data, in accordance with one embodiment of the present invention.

FIG. 7A is a photographic image of a 2-D fluoroscopic image.

FIG. 7B is a representative illustration of 3-D MRI image data acquired in the axial plane.

FIG. 7C is a representative illustration of the 3-D MRI image data of FIG. 7B reconstructed in 3-D space.

FIG. 7D is a representative illustration of the reconstructed 3-D MRI image of FIG. 7C compressed in the coronal direction.

FIG. 7E is a representative illustration of a composite image using a combination of the images of FIG. 7A and FIG. 7D, in accordance with an embodiment of the present invention.

FIG. 8 is a flow chart illustrating a method for generating the composite image of FIG. 7E, with a feature of user-selected tagging, in accordance with one embodiment of the present invention.

FIG. 9 is a flow chart illustrating a method for generating the composite image of FIG. 7E, including feature tagging, in accordance with another embodiment of the present invention.

FIG. 10 is a flow chart illustrating a method for generating a composite image using a combination of an arteriogram and a venogram, in accordance with an embodiment of the present invention.

FIG. 11 is a flow chart illustrating a method for generating a composite image using a combination of an arteriogram and a venogram, including feature tagging, in accordance with another embodiment of the present invention.

FIGS. 12A and 12B are block diagrams of a system for catheter-based renal ablation and 3-D composite imaging with catheter visualization, in accordance with an embodiment of the present invention.

FIGS. 13A and 13B are a flow chart illustrating a method for generating a 3-D composite imaging with catheter visualization for use with the system of FIGS. 12A and 12B.

FIG. 14 is a representative illustration of a method of limiting blood flow in a renal vein in the vicinity of a renal artery subject to renal denervation via catheter-based ablation.

FIG. 15 is a perspective view, with parts broken away, of a distal tip section of a catheter of the present invention, according to an embodiment.

### DETAILED DESCRIPTION OF THE INVENTION

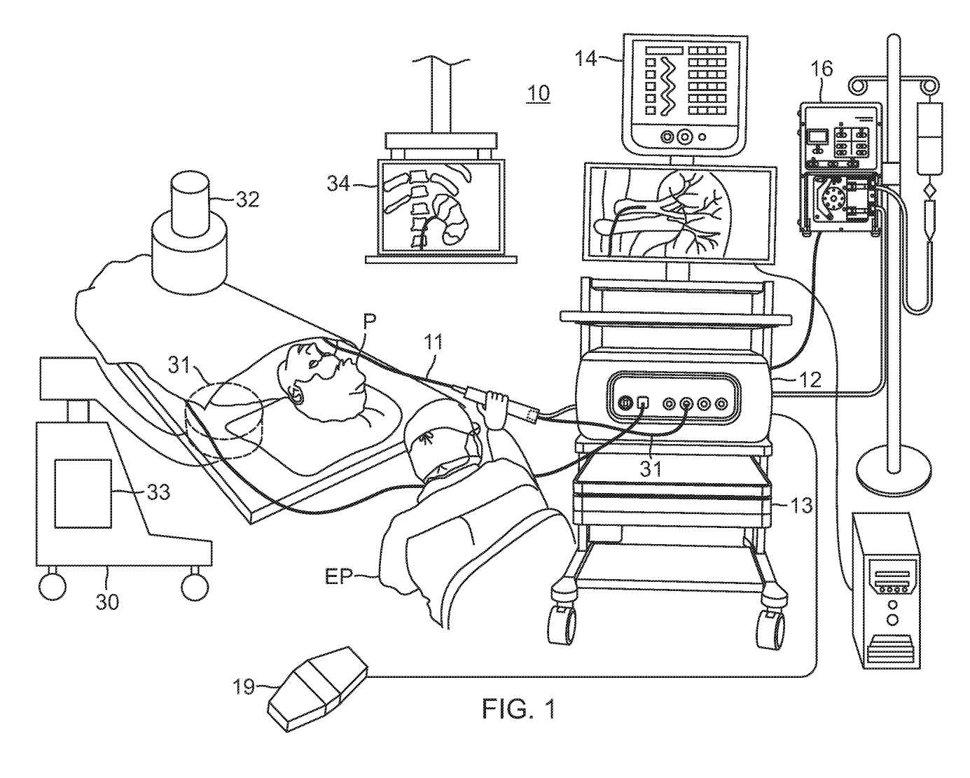

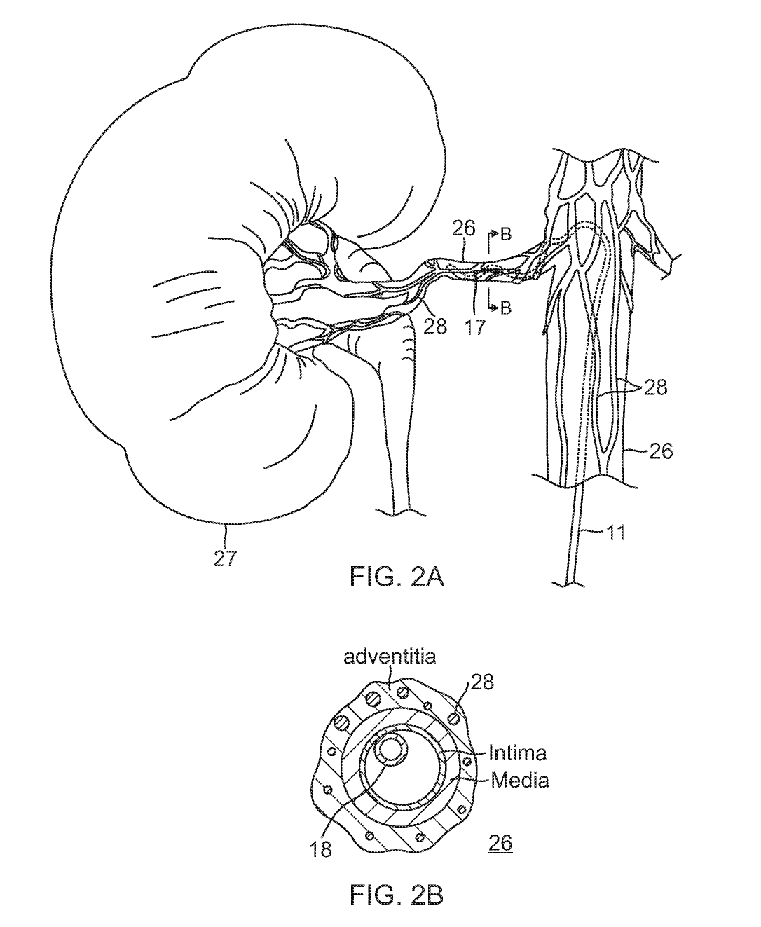

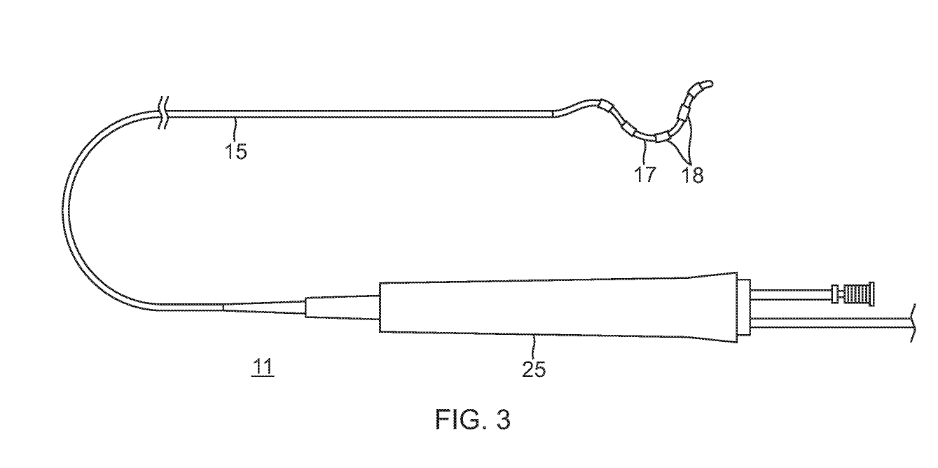

The present invention is directed to a catheter-based ablation and visualization system 10, with embodiments illustrated in FIG. 1, including a catheter 11, an RF generator console 12, a power supply 13, a first display monitor 14, an irrigation pump 16, and an ablation actuator 19 (e.g., a foot pedal). The system also includes a fluoroscopic imaging unit 30 with an X-ray source 31, a camera 32, a digital video processor 33, and a second display monitor 34. The system 10 is adapted for renal ablation performed within a renal artery 26 near a kidney 27 in denerving surrounding nerve fibers 28, as shown in FIGS. 2A and 2B. In some embodiments, as shown in FIG. 3, the catheter 11 includes a control handle 25, a catheter body 15 and a helical distal portion 17 on which electrodes 18 are mounted, each adapted for contact with a different surface area of the inner circumferential tissue along the artery 26. As known in the art, the catheter 11 enters the body of patient P in FIG. 1 via an opening in the femoral artery and is then advanced through the patient's vasculature by an electrophysiology professional EP, such as a cardiologist, under fluoroscopic guidance by the fluoroscopic imaging unit 30 and the display monitor 34, or other suitable guidance devices, to position the helical distal portion 17 in the renal artery 26 in order to ablate renal plexus nerve fibers 28 located around the renal artery 26. In some embodiments, as shown in FIG. 3, the catheter 11 has a plurality of five irrigated electrodes 18, although it is understood that the plurality may range between about three and eight.

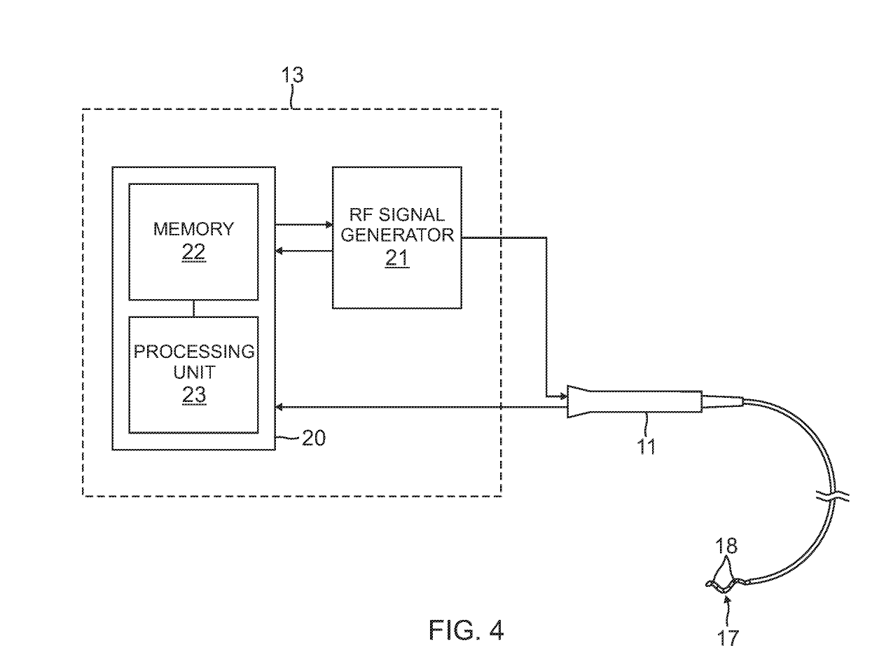

In some embodiments as shown in FIG. 4, the RF generator console 13 includes a controller 20 with memory 22 and processing unit 23, and an RF signal generator 21. The memory 22 stores instructions that, when executed by the processing unit 23, cause the controller 20 to control the RF power output by the RF signal generator 21 (e.g., by adjusting the output current) to the electrodes 18 on the catheter 11. The processing unit 23 may be any sort of computing device suitable for controlling the power output, for example, a general purpose processor coupled to a memory (e.g., dynamic random access memory and/or flash memory), a microcontroller, an appropriate programmed field programmable gate array (FPGA), or an application specific integrated circuit (ASIC).

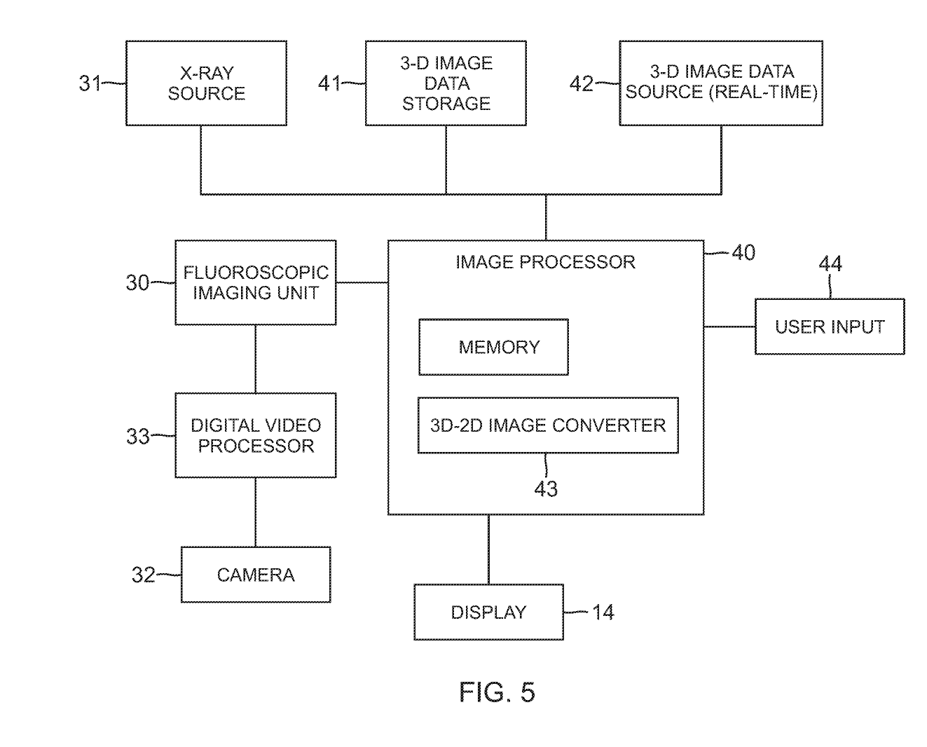

In some embodiments, as shown in FIG. 5, the system 10 includes an image processor 40 linked to the fluoroscopic imaging unit 30 to receive 2-D fluoroscopic image data captured by the camera 32 as a first image (or first image data). The image processor 40 is also adapted to receive 3-D image data which may originate from a Magnetic Resonance Imaging (MRI) system, a Computerized Tomography (CT) system, an X-ray imaging system, an ultrasonic imaging system or any other suitable imaging system or source. The 3-D image data may be acquired in advance and stored, or it may be acquired in real-time concurrently with the fluoroscopic and ablation procedure. Thus, the image processor 40 is structured and arranged to receive 3-D image data as a second image from at least the image data storage 41 or the image data source 42 which it processes with, for example, a 3D-2D image converter 43, for combination or superimposition with the 2-D fluoroscopic image to provide a composite image that is displayed on display 14. The image processor 40 is also structured and arranged to receive manual interaction via user input 44.

By combining the 2-D fluoroscopic image data which generally displays a moderate amount of visual imagery, with a more detailed visual imagery from the second image data, especially 3-D image data, such as MRI or CT scan images, a combined or composite image (used interchangeably herein) is provided to the cardiologist, not only of the catheter and selected anatomical features, such as the renal artery, visible in the fluoroscopic image, the composite image provides a more detailed image of the renal artery and advantageously of any adjacent anatomical structures from the 3-D image data, including renal veins and/or lymph nodes, that may adversely impact lesion formation. By enabling the cardiologist to view adjacent anatomical structures, whether internal or external to the renal artery, the cardiologist can be more informed on her/her selection of ablation sites, including avoidance of such adjacent anatomical structures which can absorb or otherwise divert ablation energy from the intended lesion.

Through image registration by the image processor 40, two or more sets of image data of single-modality or multi-modalities are integrated or superimposed to create a composite image data. In some embodiments, a reference or source image (e.g., fluoroscopic image) is kept generally unchanged. A sensed or target image (e.g., 3-D image) is to be spatially aligned with the reference image. A correspondence is established between a plurality of distinct points or “landmarks” in the reference and target images. By establishing the correspondence between the plurality of distinct points, a geometrical transformation can be determined to map the target image to the reference image to establish point-by-point correspondence.

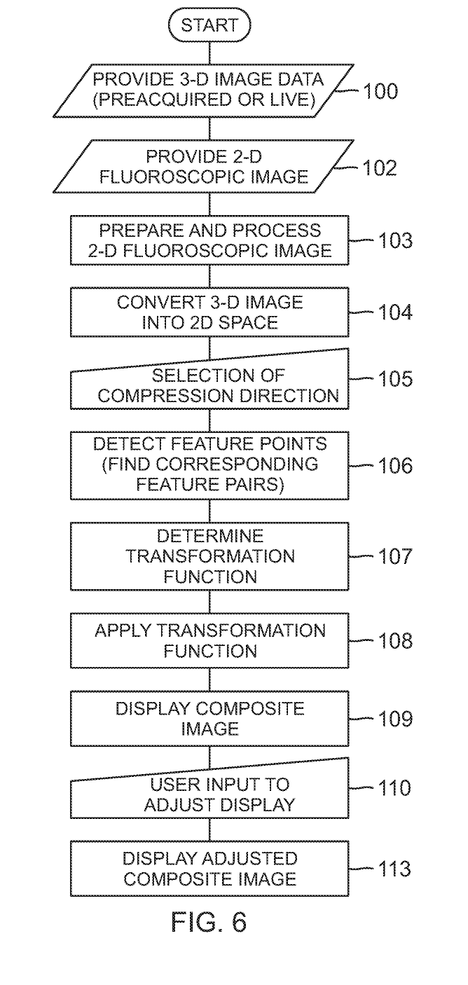

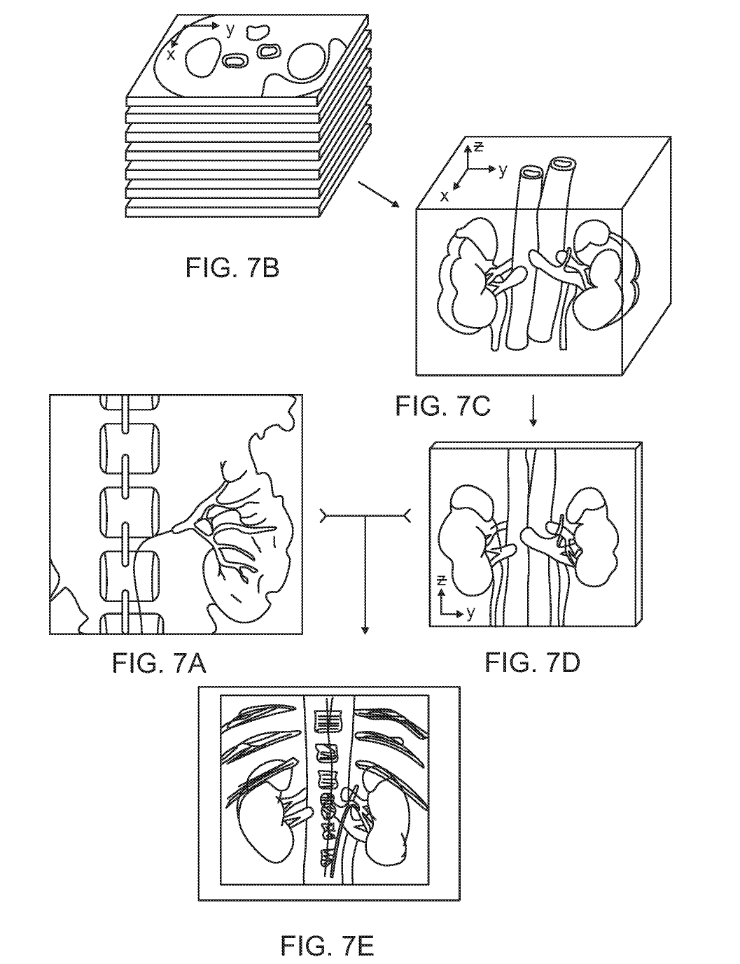

Reference is now made to FIG. 5, and to FIG. 6 which is a flow chart of a method of providing a composite anatomical image, with image registration, including feature-based registration, to spatially align the second image with the fluoroscopic image, in accordance with some embodiments of the present invention. After a user has activated the system, the user provides either a pre-acquired 3-D image data or a “live” feed of 3-D image data in real-time to the image processor 40, in Operation 100. In Operation 102, the 2-D fluoroscopic image data is provided to the image processor. In Operation 103, the image processor processes and prepares the fluoroscopic image data for superimposition, which may include assigning a coordinate system to the fluoroscopic image. In Operation 104, the image processor employs the 3D-2D image converter 43 to convert the 3-D image data into 2-D space to be compatible with the 2D fluoroscopic image (FIG. 7A). For example, the converter 43 can reconstruct the 3-D image data, originally acquired in an axial plane (FIG. 7B), in a coronal plane by creating the 3-D image data in 3-D space (FIG. 7C) and then compressing in a coronal direction (FIG. 7D), to be compatible with a coronal view of the 2-D fluoroscopic image (FIG. 7A) typical with a patient lying supine under the camera 32 of the fluoroscopic imaging unit 30 of FIG. 1. The selection of a compression direction can be made either automatically by the image processor 40 or by user input 44 in Operation 105. The compressed coronal view of FIG. 7D thus contains within a single 2-D image all the data of the 3-D image but in a direction that is compatible with the view of the fluoroscopic image of FIG. 7A. In Operation 106, the image processor detects feature points in both the 2-D fluoroscopic and compressed 2-D second image by finding corresponding feature pairs. For example, feature points may be selected as being both distinctive and invariant to properties, such as rotation, intensity and spatial scale. In Operation 107, the image processor determines a transformation function from coordinates of points of corresponding feature pairs. The transformation function may be linear or elastic/nonrigid, and single-modality or multi-modality methods, as desired or appropriate.

In Operation 108, the image processor applies the function to transform or warp the 3-D image to take the geometry of the 2-D fluoroscopic image. The Operation 108 may include resampling the second image to the coordinate system of the fluoroscopic image using the transformation function. In Operation 109, the image processor displays a composite image including the fluoroscopic image and the 3-D image superimposed (FIG. 7E). In Operation 110, the cardiologist may adjust the composite image, for example, by changing the scale and/or pan in/out of the composite image, as desired, via the user input 44. In Operation 113, the image processor displays the adjusted composite image on display 14.

Accordingly, the composite image (FIG. 7E) includes details provided by the 3-D image data which are not present or not highly visible in the fluoroscopic image. Thus, anatomical structures otherwise not shown or visible in the fluoroscopic image on display 34 are shown or visible in the composite image on display 14. For example, renal veins and/or lymph nodes present or visible in the second image but not present or highly visible in the fluoroscopic image on display 34 are now present or visible in the composite image on display 14, so that the cardiologist can intelligently position the catheter in the renal artery in avoiding anatomical structures that may adversely affect lesion formation.

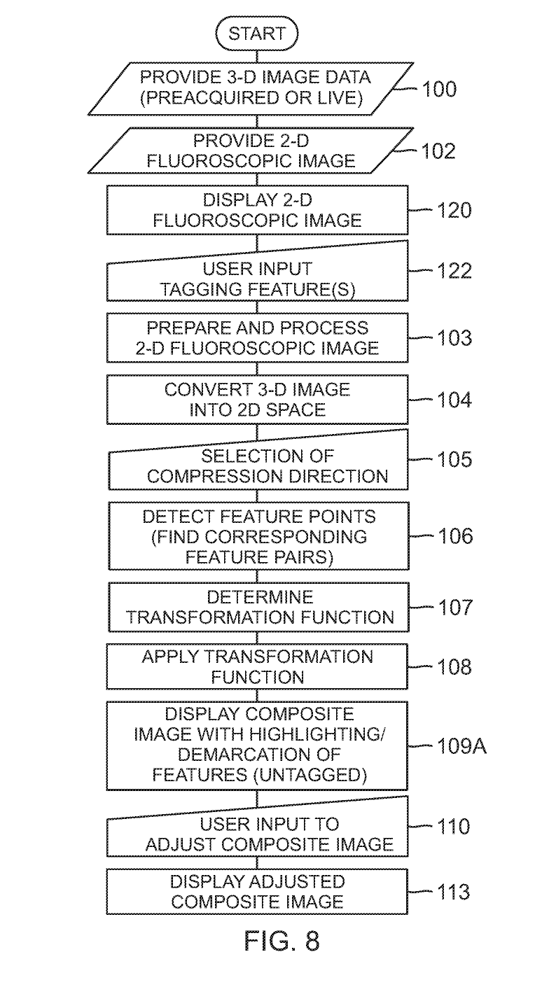

In some embodiments of the system, as shown in FIG. 8, one or more operations involving manual interactive methods are provided to highlight selected anatomical structures in the composite image, including renal veins and/or lymph nodes. For example, in operation 120, the image processor displays the fluoroscopic image on the display monitor and enables the cardiologist in operation 122 to manually mark (or “tag”) selected features visible in the fluoroscopic image, such as the renal artery, or feature portions thereof, where such tagged features may be used as “landmarks” in the superimposition process.

The image processor then detects features in the compressed 2-D image that correspond with such tagged features, which may be part of or separate from the processes of the Operations 106, 107 and 108. In operation 109A, the image processor provides a composite image wherein features not tagged in the fluoroscopic image, such as renal veins and/or lymph nodes, including, for example, those adjacent the tagged features, are visually enhanced (for example, with greater intensity, different color and/or outlining), so that the cardiologist can have better viewing of the adjacent features he may wish to avoid and can better position the catheter in regions of the renal artery that are more distanced or remote from the visually enhanced anatomical structures, including renal veins and/or lymph nodes, which can adversely impact lesion formation.

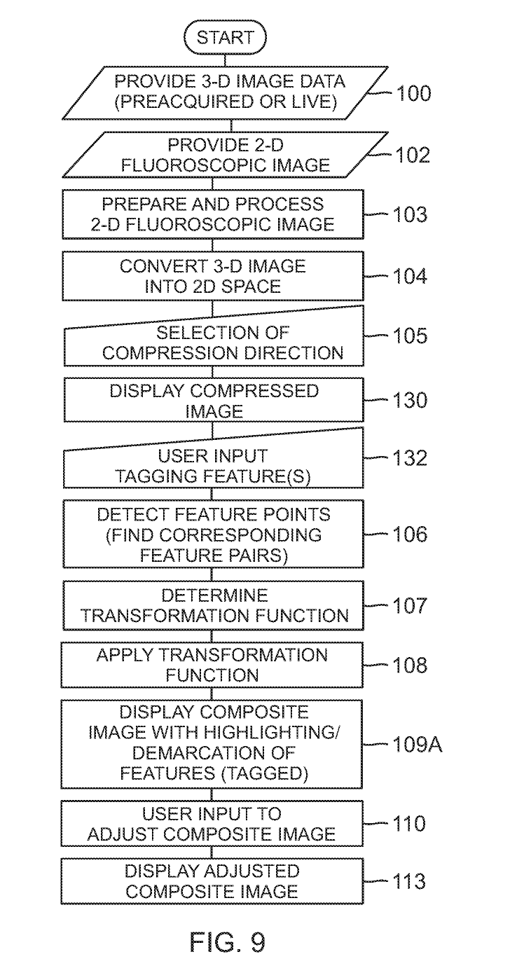

In some embodiments of the system, as shown in FIG. 9, wherein selected anatomical structures are visually enhanced in the composite image involving manual interactive methods, the image processor, in Operation 130, displays the compressed 2-D image on the display monitor 14 and enables the cardiologist in Operation 132 to manually mark (or “tag”) selected anatomical structures visible in the compressed 2-D image but not present or highly visible in the fluoroscopic image, such as the renal veins and lymph nodes, or feature portions thereof. The image processor then proceeds with Operations 105, 106, 107 and 108, and in Operation 109A provides a composite image wherein structures tagged in the compressed 2-D image, such as renal veins and/or lymph nodes, including, for example, those adjacent the tagged features, are visually enhanced (for example, with greater intensity, different color and/or outlining), so that the cardiologist can have better viewing of the features he may wish to avoid which can adversely impact lesion formation.

The cardiologist may use a pointing device, a mouse, a touch-sensitive screen or tablet coupled to the display monitor, or any other suitable input device. The combination of the display and pointing device is an example of an interactive display, i.e., means for presenting an image and permitting the user to mark on the image in such a way that a computer is able to locate the marks in the image. Other types of interactive display will be apparent to those skilled in the art.

The some embodiments, tagging may be conducted in a semi-automatic manner. For example, the image processor may run suitable feature detection software that automatically tags detected features in the first and/or second images. The cardiologist then reviews and edits the automatically-detected tagged features using the interactive display.

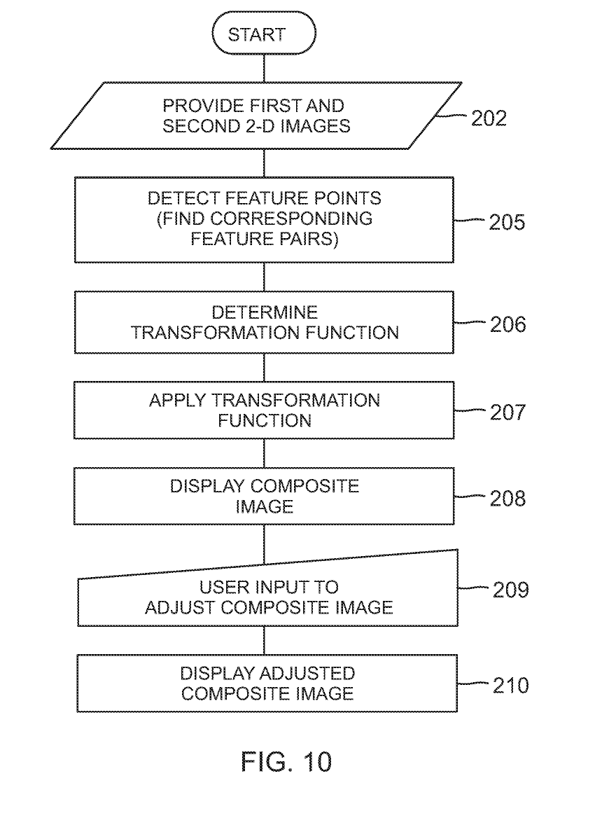

Reference is now made to FIG. 10, which is a flow chart of a method of providing a composite image, with image registration to spatially align first and second fluoroscopic images, including, for example, a 2-D arteriogram and a 2-D venogram, in accordance with some embodiments of the present invention. Angiograms are 2-D X-ray images of vasculature which contain an injected dye or “contrast” so that blood flowing in the vasculature is visible on X-rays. Arteriograms are angiograms of arteries and venograms are angiograms of veins. Different parts of the body absorb the x-rays in varying degrees. Dense bone absorbs much of the radiation while soft tissue, such as muscle, fat and organs, allow more of the x-rays to pass through them. As a result, bones appear white on the x-ray, soft tissue shows up in shades of gray and air appears black. Visible soft tissue abdominal organs include the liver, spleen, kidneys, psoas muscles, and bladder. Accordingly, bones and these soft tissues are often also visible on angiograms in addition to the arteries or veins of interest.

After an cardiologist has activated the system, in Operation 202, the image processor receives a first image, e.g., an arteriogram, of a renal region and, a second image, e.g., a venogram, of the renal region, wherein one or both of the angiograms are provided in real-time or are pre-acquired. In Operation 205, the image processor detects feature points in both images by finding corresponding feature pairs. For example, feature points may be selected as being both distinctive and invariant to properties, such as rotation, intensity and spatial scale.

In Operation 206, the image processor determines a transformation function from coordinates of points of corresponding feature pairs. The transformation function may be linear or elastic/nonrigid, and single-modality or multi-modality methods, as desired or appropriate. In Operation 207, the image processor applies the function to transform or warp the second image to take the geometry of the fluoroscopic image. The Operation 207 may include resampling the second image to the coordinate system of the first image using the transformation function. In Operation 208, the image processor displays a composite image where the first and second images are registered, and the composite image includes both the artery/arteries and/or arterial features visible in the arteriogram and the vein/veins and/or venous features visible in the venogram, along with any catheter(s) positioned in the renal region. In Operation 209, the cardiologist may adjust the composite image, for example, by changing the scale and/or pan in/out of the composite image, as desired, via the user input 42. In Operation 210, the image processor displays the adjusted composite image in which features from the arteriogram and features from the venogram are visible.

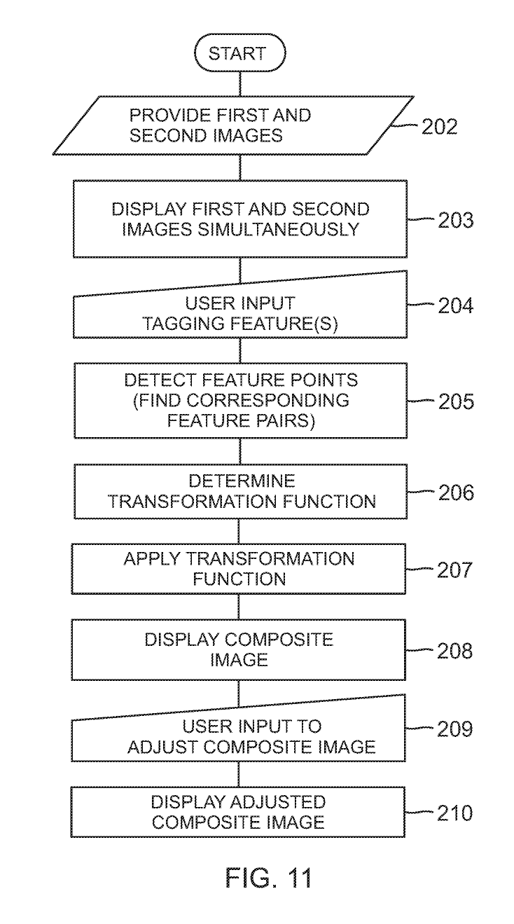

In some embodiments, as shown in FIG. 11, in Operation 203, the first and second images are displayed on the display monitor 14, for example, simultaneously, in side-by-side format. In Operation 204, the cardiologist may “tag” corresponding features, e.g., bone and soft tissue, including ribs, kidneys, liver, lymph nodes, and the like, present or visible in both images. In Operations 205, 206 and 207, the image processor uses the tagged features as “landmarks” to register the first and second images. In Operation 208, the image processor provides a combined or composite image that includes both the artery/arteries and/or arterial features visible in the arteriogram and the vein/veins and/or venous features visible in the venogram, along with any catheter(s) positioned in the renal region. As such, the cardiologist can identify one or more veins adjacent the artery or arteries which he/she may wish to avoid during ablation and be better informed as to where he/she may wish to position the catheter in the artery or arteries.

In some embodiments, the cardiologist may in Operation 209 “tag” any feature(s) in the composite image generated from Operation 208 to be visually enhanced (or visually diminished) in the adjusted composite image generated from Operation 210.

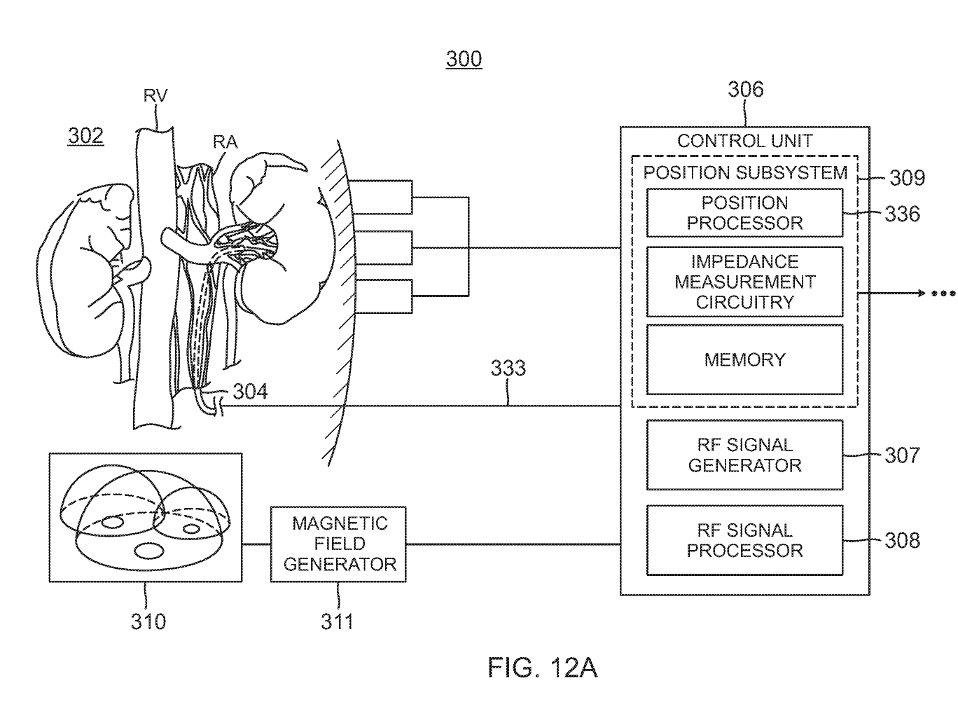

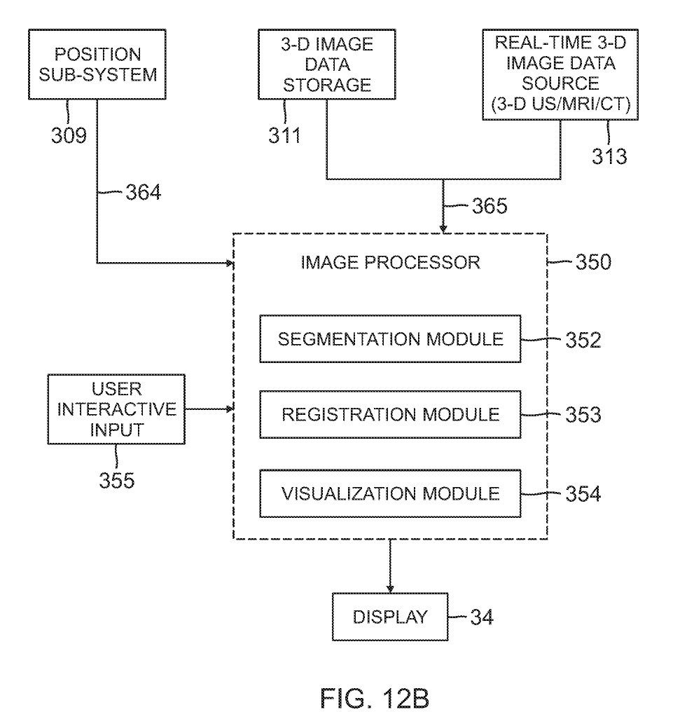

FIGS. 12A and 12B are block diagrams of a system 300 for mapping and visualizing the renal region 302 of a patient, in accordance with some embodiments of the present invention. The system comprises a catheter 304, which is inserted by a physician into the renal vasculature, for example, a renal artery RA. Catheter 304 typically comprises a handle for operation of the catheter by the physician. Suitable controls on the handle enable the physician to steer, position and orient the distal end of the catheter as desired.

System 300 comprises a controller 306 having an RF signal generator 307, an RF signal processor 308 for enabling catheter ablation. The controller 306 includes a position sub-system 309 that measures position (including location and orientation coordinates) of catheter 304 and generates 3-D mapping data. (Throughout this patent application, the term “location” refers to the spatial coordinates of the catheter, and the term “orientation” refers to its angular coordinates. The term “position” refers to the full positional information of the catheter, comprising both location and orientation coordinates.) In one embodiment, the position sub-system 309 utilizes magnetic position tracking to determine the position and orientation of catheter 304 for visualization on the display monitor 14.

Mapping of a renal region typically involves using a mapping catheter with a position sensor with electrodes to record electrical activity in the region of interest. The XYZ location of the data points are used to create and refine the geometry of the chamber being mapped. Called “point by point” mapping, a cardiologist “builds out the shell” as he acquires more and more points. The catheter is moved along the wall of the anatomical structure to record location points to generate 3D anatomical geometry. Through acquiring new points a three-dimensional anatomic map is created or developed in real-time. Moreover, sites or locations of anatomical relevance may be recorded or “tagged”. A reference patch (not shown) is affixed to the patient's back roughly overlying the region of interest. This allows for accurate tracking of the mapping catheter position, consistency of anatomic landmark and precise reconstruction of anatomical geometry. More recently, Fast Anatomical Mapping (FAM), a feature on the CARTO 3 mapping system, permits rapid creation of anatomical maps simply by the movement of a magnetic location sensor-based catheter throughout the anatomical region. The cardiologist can create the 3-D anatomical map or “shell” of the region of interest as rapidly as he can move the catheter along the wall of the region.

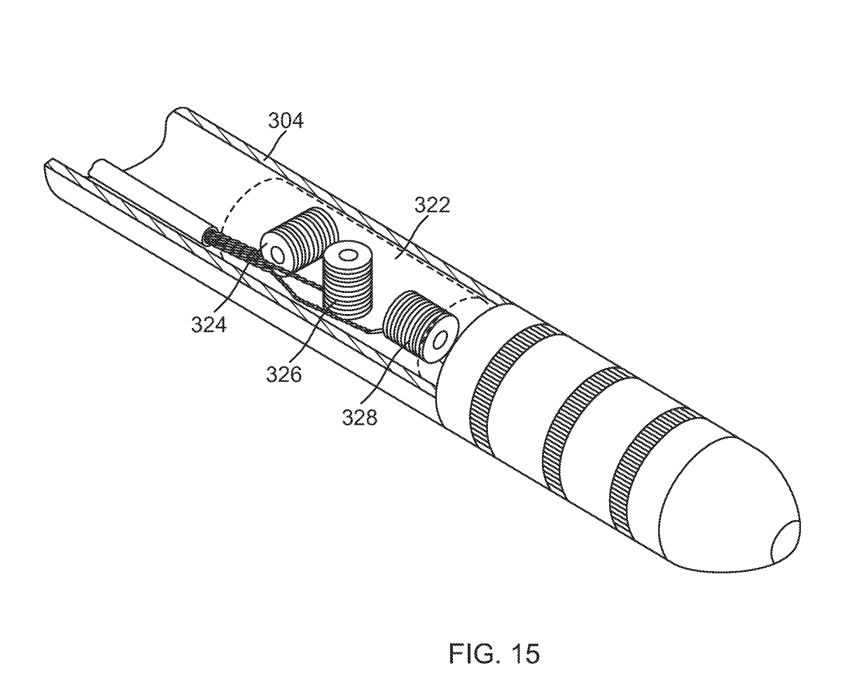

The position sub-system 309 generates magnetic fields in a predefined working volume within its vicinity and senses these fields at the catheter. The position sub-system typically comprises a set of external radiators, such as field generating coils 310, which are located in fixed, known positions external to the patient. Coils 310, driven by a magnetic field generator 311, generate fields, typically electromagnetic fields, in the vicinity of renal region 302. The generated fields are sensed by a position sensor 322 (comprising three orthogonal sensing coils 324, 326 and 328) inside a distal tip section of the catheter 304, as shown in FIG. 15. In an alternative embodiment, a radiator, such as a coil, in the catheter generates electromagnetic fields, which are received by sensors outside the patient's body. The position sensor 322 transmits, in response to the sensed fields, position-related electrical signals over cable 333 (FIG. 12A) running through the catheter to the position sub-system 309. Alternatively, the position sensor may transmit signals to the position sub-system 309 over a wireless link. The sub-system 309 comprises a position processor 336 or “workstation” that calculates the location and orientation of catheter 304 based on the signals sent by position sensor 322. Position processor 336 typically receives, amplifies, filters, digitizes, and otherwise processes signals from catheter 304. Some position tracking systems that may be used for this purpose are described, for example, in U.S. Pat. Nos. 6,690,963, 6,618,612 and 6,332,089, and U.S. Patent Application Publications 2002/0065455 A1, 2004/0147920 A1 and 2004/0068178 A1, whose disclosures are all incorporated herein by reference. Although the position sub-system 309 uses magnetic fields, the methods described herein may be implemented using any other suitable positioning sub-system, such as systems based on electromagnetic fields, acoustic or ultrasonic measurements, to generate 3-D mapping data.

The position sub-system 309 may also include visualization of “non-sensor-based catheters” present in the region of interest in generating 3-D catheter visualization data. Such catheter visualization may display localized electrodes of those catheters, wherein “localization” (location/position detection of electrodes) is obtained through impedance or current-based measurements. For example, impedance is measured between electrodes affixed to the catheter and electrodes placed on the body surface. The position of the catheter and its electrodes is then derived from the impedance measurements. Methods for impedance-based position sensing are disclosed, for example, in U.S. Pat. No. 5,983,126 to Wittkampf, in U.S. Pat. No. 6,456,864 to Swanson, and in U.S. Pat. No. 5,944,022 to Nardella, the entire disclosures of which are incorporated herein by reference.

Thus, the two methods of catheter visualization with a patient's body use sensor-based and nonsensor-based catheters. Sensor-based catheters use sensors inside the catheter tip to measure the relative strengths of externally-generated magnetic fields and triangulate the location and orientation of the catheter. In contrast, the location and orientation of non-sensor-based catheters are derived from current or impedance measurements between the catheter's own electrodes and externally placed electrodes. Accordingly, the position sub-system 309 provides at least 3-D mapping data of a mapped anatomical region by which the anatomical region can be reconstructed in 3-D, and 3-D catheter visualization data of the location and orientation of a catheter by which the catheter can be visualized.

The CARTO 3 mapping system, available from Biosense Webster, Inc., employs a hybrid technology of both the magnetic location sensing and current-based data to also provide visualization of both sensor-based and non-sensor-based catheters and their electrodes. The hybrid system, called the Advanced Catheter Location (ACL) feature, is described in U.S. Pat. No. 7,536,218 to Govari et al., the entire disclosure of which is incorporated herein by reference. The ACL technology is responsive to movement of the electrodes of the catheters and therefore updates the image of the electrodes in real time to provide a dynamic visualization of the catheters and their electrodes correctly positioned, sized, and oriented to the displayed map area on the CARTO 3 mapping system. The catheter visual representations therefore respond to being repositioned by the physician, dislodging from position, and subtler movements such as those caused by the patient's own breathing pattern. This dynamic movement of the catheter images stands in contrast to the 3-D maps themselves, which are created from a set of recorded locations and are, thus, static.

As will be explained and demonstrated below, system 300 of FIGS. 12A and 12B enables the cardiologist to perform a variety of mapping and imaging procedures. As shown in FIG. 12B, the system 300 processes and integrates the 3-D mapping data, the 3-D catheter visualization data from the position-sub-system 309, and 3-D image data (pre-acquired or from a “live” feed in real-time) further includes an image processor 350 coupled to the position sub-system 309 and a 3-D image data storage 311 and a 3-D image recording system 313 that is adapted to receive 3-D image data from one or more tomographic image sources, for example, MRI, CT-scan and/or 3-D ultrasound devices. The image processor 350 includes a segmentation module 352, a registration module 353 and a visualization module 354. The image processor 350 is structured and arranged to drive the display monitor 34 to display generate a composite 3-D image including anatomical geometries from both the 3-D mapping data and the 3-D tomographic image data, including anatomical geometries not present or visible in the 3-D mapping data. By utilizing the aforementioned hybrid technology, the image processor 350 also incorporates movement of the electrodes of the catheters and therefore updates the image of the electrodes in real time to provide a dynamic visualization of the catheters and their electrodes correctly positioned, sized, and oriented to the displayed anatomical region.

These procedures comprise, for example, the following: Acquire a first image data, including 3-D mapping data of one or more anatomical geometries of a selected region. Import a second image data, including pre-acquired or live feed in real-time 3-D image data (e.g., recorded by way of a method of tomographic 3D imaging, such as X-ray computer tomography, MRI tomography or 3-D ultrasonic techniques, that include at least a portion of the anatomical geometries of the selected, and additional anatomical geometries within the selected region and/or additional anatomical geometries outside of the selected region. Segment the 3-D image data to extract surface profile of the one or more anatomical geometries. Register and generate a composite 3-D image set containing anatomical geometries from both 3-D mapping data and the 3-D image data, including anatomical geometries not present or visible in the 3-D mapping data. Display the composite 3-D image.

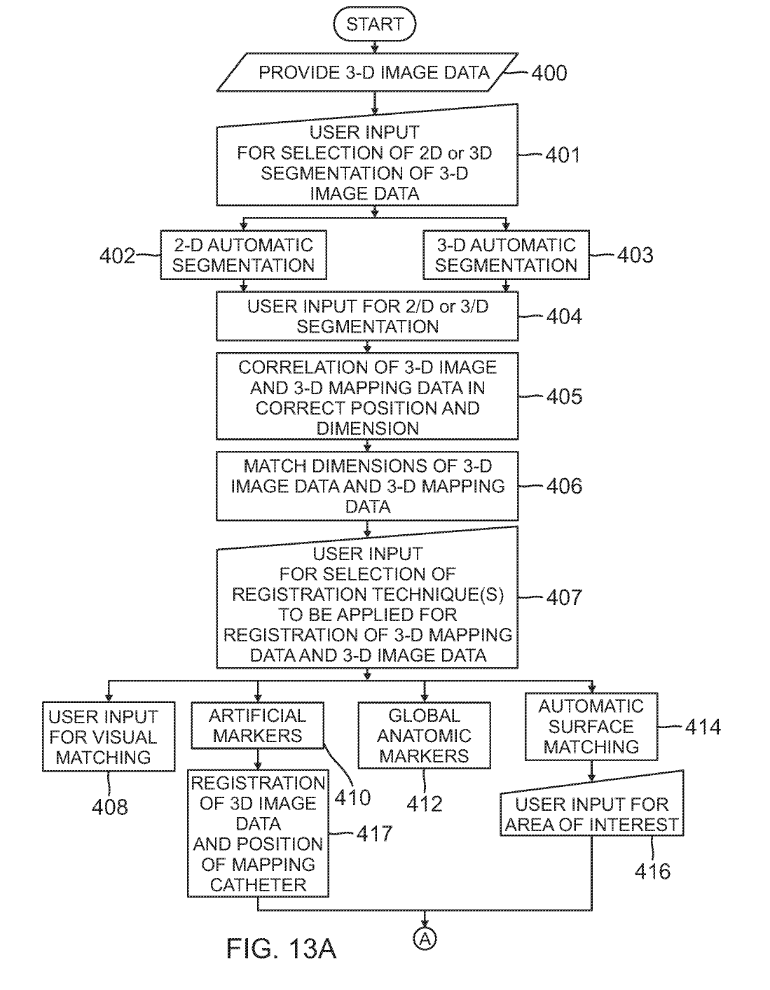

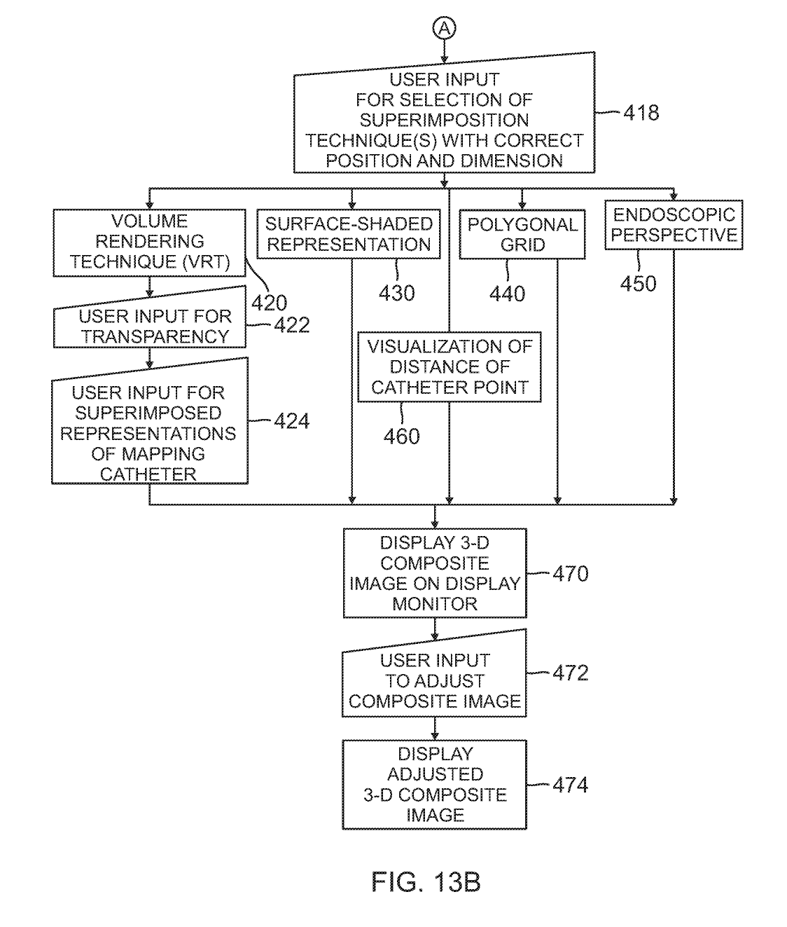

Reference is now made to FIGS. 13A and 13B, which is a flow chart of a method of providing a composite image. In Operation 400 in the present method, the 3D image data of the area to be treated, particularly of the renal vessel to be treated, are recorded, or if pre-acquired are imported to the image processor. During the recording of these 3D image data, a larger portion of the renal vessel and/or its surrounding tissues can also be included for the registration to be performed later. The 3D image data are recorded by way of a method of tomographic 3D imaging such as, for example, X-ray computer tomography, magnetic resonance tomography or 3D ultrasonic techniques.

During the performance of the method in some embodiments, it may be preferable to record high-resolution image data of one or more renal vessels. Preferably, a contrast medium in association with a test bolus or bolus tracking is therefore used for recording the 3D image data.

The 3D image data is segmented for extracting the 3D surface profile of the renal vessel. This segmentation is employed, on the one hand, for the later representation of the surface profile of these objects in the superimposed image representation and, on the other hand, in an advantageous embodiment of the method, for correlation with the 3D mapping data in the correct position and dimension.

The segmentation takes place in the segmentation module 352 of the system 300 (FIG. 12B). This segmentation module 352 receives the recorded 3D image data via a corresponding input interface 364. In the same way, the 3D mapping data are supplied to the image processor 350 via the same or another interface 365, as a general rule in some embodiments continuously, during the period of the electrophysiological catheter application.

The segmentation of the 3D image data can be applied in the same manner to one or more regions of the renal vessel in order to obtain all surfaces which are represented by the 3D mapping data. However, a registration by surface matching does not require segmentation of the entire surface or of the renal vessel to be treated, respectively. For this, it is sufficient to obtain a representation of the surface of an area of the renal vessel, for example the renal artery, at a few surface points by which the surface matching can be performed for the registration. On the other hand, it may be advantageous to include a larger area, particularly further vessels, for the registration.

The segmentation of the renal vessel to be treated can take place in the form of a 2D segmentation in individual layers or in the form of 3D segmentation. One possibility resides in Operation 402 performing a fully automatic segmentation of all layers of the renal vessel obtained by the imaging method. As an alternative, one or more of the layers can also be segmented interactively in Operation 404 by the cardiologist operator via user interactive input 355 (FIG. 12B) and the layers following in each case can be segmented automatically on the basis of the prior knowledge of the layers already segmented. The interactive segmentation of individual layers can also be supported by semi-automatic techniques such as, for example the technique of active contours. After the segmentation of all individual layers, the 3D surface profile of the renal vessel can then be reconstructed.

The segmentation can also take place as 3D segmentation of the renal vessel to be treated by way of known 3D segmentation techniques in Operation 403. Examples of such 3D segmentation techniques are the threshold technique or the technique of region growing. If these fully automatic 3D segmentation algorithms do not work reliably in individual cases, the cardiologist can in Operation 404 specify, for example, gray scale thresholds or spatial blockers, via the user interactive input 355.

Accordingly, the process of FIGS. 13A and 13B may include in Operation 401 receiving a selection by the cardiologist to process 3-D image data with 2-D segmentation or 3-D segmentation.

The 3D surface profile of the objects, obtained from the segmentation, is supplied to the registration module 353 (FIG. 12B) in which the 3D image data or, respectively, the data of the 3D surface profile obtained from these, are correlated with the 3D mapping data in Operation 405 in the correct position and dimension. The 3D mapping data are obtained via a mapping catheter which supplies 3D coordinates of surface points of the renal vessel to be treated via a 6D position sensor integrated into the tip of the catheter. Such catheters are known from the prior art for catheter ablation or, respectively, electro-anatomical mapping.

In this process, the catheter is introduced into the respective renal vessel by the cardiologist. During the catheter mapping, increasingly more surface points are added to the mapping data in the course of time. These surface points are used for reconstructing the morphological structure of the renal vessel, i.e. for visualizing it. In this manner, an increasingly more detailed image of the renal vessel to be treated is produced from the 3D mapping data in the course of time.

In Operation 406 in the registration module 353, dimensions of the 3D image data and of the 3D mapping data are also matched apart from the correlation in Operation 405 in the correct position. This is used in some embodiments in order to achieve the most accurate superimposition possible of the 3D image data of the renal vessel or of its surface in the same position, orientation, scaling and shape with the corresponding visualization of the renal vessel from the 3D mapping data.

As a general rule in some embodiments, this uses a transformation of the 3D image data or of the 3D mapping data which can include three degrees of freedom of translation, three degrees of freedom of rotation, three degrees of freedom of scaling and/or a number of vectors for the deformation.

In some embodiments, the registration can take place by visual matching. For this purpose, the cardiologist in Operation 408 changes the data visualized until the position, orientation, scaling and/or shape of the renal vessel displayed matches in both representations, i.e. on the basis of the 3D image data and on the basis of the 3D mapping data. The visual matching can take place via the user interface input 355.

Furthermore, artificial markers can be used for the registration in Operation 410. In some embodiments, the artificial markers can thus be attached to the torso of the patient before recording the 3D image data. These markers remain fixed at the same position during the entire subsequent catheter application. At least three of these markers are used for achieving correct registration, i.e. correlation of the image data with the mapping data. During this process, markers used are both recognizable in the 3D image data and identifiable by the position sensor of the mapping system.

Other further embodiments for registration provide the use of global anatomic markers, i.e. distinct natural points of the area to be treated or its environment, for a registration in Operation 412. These distinct points must be identifiable in the 3D image data and are preferably approached with the mapping catheter by using a fluoroscopic imaging technique. Such distinct points are, for example, bifurcations, the aorta, the renal veins and the kidneys themselves. The distinct points can then be detected automatically in the 3D image data and the 3D mapping data so that a correlation of these data with the correct position and dimension can be calculated.

In addition, a registration between the position of the mapping catheter and of the 3D image data can also be carried out via such markers or distinct points in Operation 417. This registration makes it possible to visualize the position of the mapping catheter within the 3D image data.

A further advantageous possibility for the registration of the 3D image data and of the 3D mapping data resides in Operation 414 in the automatic matching of the surfaces represented on the basis of these data. After the segmentation of the renal vessel(s) to be treated, the extracted 3D surface contour of the renal vessel can be automatically matched to the surface contour of the renal vessel obtained by the 3D mapping data. In the case of deviations in the shape of the surface contours obtained from the 3D image data and the 3D mapping data, deforming matching algorithms can be applied to the surface contour from the 3D image data or to the surface contour from the 3D mapping data in order to improve the mutual mapping.

The surface matching can be performed, for example, by reducing or even minimizing point spaces between surface points of the mapping data and surface points of the 3D surface contour extracted from the 3D image data (point-to-point matching). As an alternative, the matching can also be performed by reducing or even minimizing point spaces between surface points of the mapping data and interpolated matching points of the 3D image data (point-to-surface matching).

The surface matching requires a good surface representation by the 3D mapping data of the renal vessel to be treated. However, since these data may be collected over a relatively long period of time, as a general rule, i.e. only a few anatomical 3D mapping data are available at the beginning of the mapping and/or ablation, a multi-stage process of the registration is preferably performed. In this process, a registration by a marker takes place in an initial first stage. The accuracy of the registration is then improved in the course of the process by surface matching in a second step.

Naturally, further steps of surface matching, by which a further increase in accuracy is possibly provided, can also be performed with the increasing number of mapping points. This multi-stage registration is advantageous since registration by surface matching, with a correspondingly good surface representation, is more accurate than registration by means of anatomical distinct points or artificial markers, but a good surface representation is only obtained in a later course of the method by the mapping data.

In the initial first stage, a combination of registration by way of markers via Operation 410 and/or Operation 412 and of registration by way of surface matching via Operation 414 can also be effected. Thus, for example, registration of a portion of the renal vessel by surface matching and registration of another portion of the renal vessel by distinct anatomical points can be effected.

A further possibility for the registration by way of surface matching in Operation 414 includes not using for the matching the surface of the renal vessel to be treated but the surface of another renal vessel which has already been anatomically measured before the beginning of the catheter application. Naturally, measuring should take place with a sufficient number of surface points in this case. The resultant matching parameters for this renal vessel can then be applied to the data obtained during the catheter ablation.

In the preceding example embodiments, the surface matching of Operation 414 was implemented as point-to-point or point-to-surface matching. Since the procedure of catheter ablation is performed on certain relatively small areas of the renal vessel to be treated, surface matching in these areas of interest provides more accurate results than in other areas of the vessel to be treated, due to the high density of mapping points. Higher weighting of surface points located within the area of interest achieves better spatial matching in this area than in other areas. The area of interest can be specified, for example, by a corresponding input by the cardiologist in Operation 416 at the graphical user interface 355.

Apart from this anatomic area of interest, surface points in the immediate vicinity of the catheter or its known position can be used for performing local surface matching. The higher weighting of these points results in better local matching around the catheter point than in other areas of the chamber to be treated. However, this method uses real-time registration during the catheter application in order to be able to continuously update the surface matching during the movement of the catheter.

Recognizing that one or more registration techniques may be implemented, Operation 407 receives selection by the cardiologist of the registration technique(s) to be implemented. After the registration between the 3D mapping data and the 3D image data, superimposition with the correct position and dimension for visualizing the superimposed data is performed in the visualization module 354. It is understood that refining the registration or superimposition during the catheter ablation may occur by way of a multi-stage process.

For the superimposed visualization, which can take place, for example, on the display monitor 14, different techniques can be used. In Operation 418, the process receives selection by the cardiologist of which superimposition technique(s) are to be implemented. In some embodiments, the visualization of the 3D image data or of the renal vessel to be treated, respectively, can be effected by means of a volume rendering technique (VRT) in Operation 420. On the image data visualized by way of the volume rendering technique, the complete 3D mapping data can be superimposed which both show electrical activity and the instantaneous position of the catheter with spatial resolution. The transparency of both part-images, i.e. the part-image from the 3D image data and the part-image from the 3D mapping data, like the blending factor of the superimposition, can be changed by the cardiologist in Operation 422 in order to obtain suitable visualizations of the anatomy, the electrophysiology or simultaneously of both characteristics. Since the visualization of the 3D mapping data contains the visualization of the position and orientation of the mapping catheter, it is also possible only to superimpose the representation of the position and orientation of the mapping catheter on the 3D image data from time to time in Operation 424.

In a further embodiment, the surface extracted from the 3D image data can also be visualized as surface-shaded representation in Operation 430 or, after triangulation, as polygonal grid in Operation 440. The polygonal grid is displayed together with the 3D mapping data in order to be able to visualize simultaneously the anatomy represented by the polygonal grid and the electrophysiology represented by the 3D mapping data. In this case, too, it is possible only to display the position and orientation of the mapping catheter together with the polygonal grid representing the surface, from time to time.

In a further embodiment, an endoscopic perspective can also be calculated from the recorded data and visualized by superimposing the anatomical 3D image data and electrophysiological 3D mapping data in Operation 450. By way of this endoscopic perspective, from the point of view of the tip of the catheter, the catheter can also be guided by the operator to the corresponding anatomical or ablation targets.

Furthermore, the recorded data can also be used for visualizing the distance of the catheter point from predeterminable areas in Operation 460. Since during the registration between the 3D mapping data and 3D image data, or during the registration between the position of the mapping catheter and the 3D image data, a spatial relation is obtained between the mapping catheter and the 3D image data, the distance of the helical distal portion 17 of the catheter from predeterminable picture elements of the 3D image can be calculated at any time. This registration makes it possible to display the mapping catheter within the representation of the 3D image data and at the same time to specify the distance.

Thus, for example, the distance of the catheter point from the target tissue can be visualized in real time in the representation. The visualization can take place, for example, by color representation of the catheter with color coding of the distance. This possibility of catheter representation can be used for planning and controlling ablation processes. Furthermore, due to the registration between the mapping catheter and the 3D image data, it is also possible to store the position of removed locations together with the image data. The position stored can be processed for documentation purposes and for the planning and control of subsequent ablation processes.

In Operation 470, the processed image data is displayed on the display monitor 34 as a 3-D composite image of one or more anatomical features present or visible in the 3-D mapping data and one or more anatomical features not present or visible in the 3-D mapping data but present or visible in the 3-D image data.

In Operation 472, the cardiologist may “tag” one or more anatomical features for visual enhancement or diminishment in the composite image. For example, the cardiologist may tag anatomical features he/she wishes to avoid during ablation. In Operation 474, the composite image is displayed on the display monitor 14.

Suitable methods of registration, segmentation, correlation, registration and superimposition are described in U.S. Pat. No. 9,078,567, the entire contents of which are incorporated herein by reference.

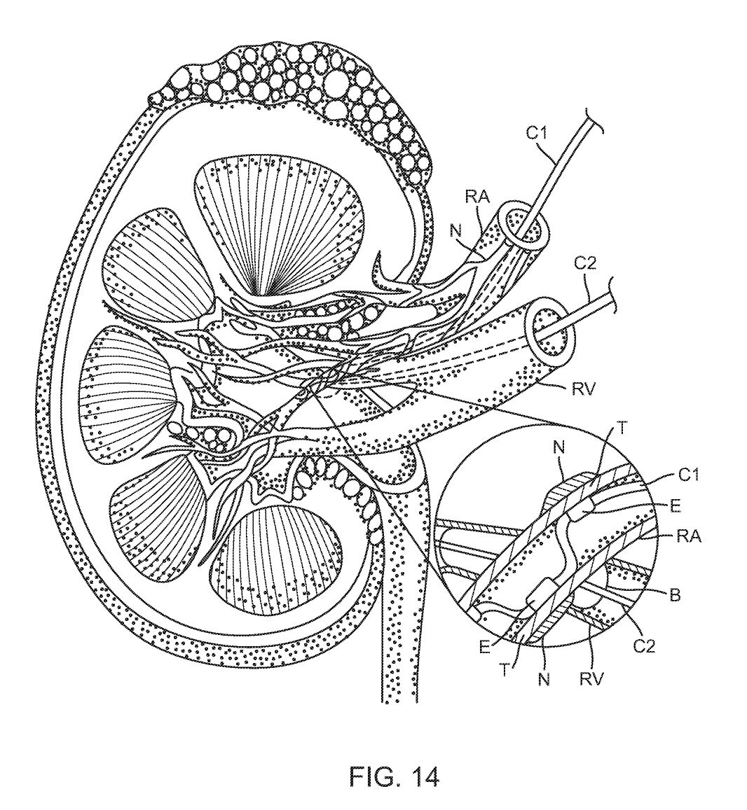

In use, the system and method of the present invention, according to some embodiments, include an cardiologist viewing the composite image on the display monitor prior to and/or during a renal ablation procedure and positioning the ablation catheter, and its ablation electrodes, with consideration of a target ablation site of a renal artery along with any adjacent anatomical structure that may adversely affect lesion formation. Such adjacent anatomical structure may include a renal vein, a lymph node or other soft tissue that may divert heat from the target ablation site. The cardiologist may adjust the catheter to reposition one or more electrodes to a new target site. Where the target site is adjacent a renal vein, the cardiologist may utilize a balloon catheter deployed in the renal vein to restrict the blood flow in the renal vein to prevent blood flow from dissipating heat from the ablation site. As shown in FIG. 14, balloon B of catheter C2 may be positioned in renal vein RV anywhere upstream of or at ablation target site T in the renal artery RA, and inflated to restrict or block blood flow in the renal vein RV adjacent the target site T. Ablation electrodes E carried on ablation catheter C1 are positioned to ablate one or more target sites in forming lesions for denervation of renal nerves N that extend around the renal artery RA. The balloon B can reduce the cooling effect of blood flow by obstruction with inflation in the renal vein, but in addition to or in lieu of such obstruction, the balloon may receive a fluid, for example, one or more gases and/or liquid substances, having a suitable temperature to help reduce the cooling effect of blood flow by reducing heat-drawing ability of the blood flow. Other suitable flow-limiting catheters include catheters with shields or umbrella-shaped devices that can be deployed to restrict, limit or block blood flow in the renal vein.

For any of the embodiments disclosed herein, the position and image processors may be implemented using a general-purpose computer, which is programmed in software to carry out the functions described herein. The software may be downloaded to the computer in electronic form, over a network, for example, or it may alternatively be supplied to the computer on tangible media, such as CD-ROM. The position processor and image processor may be implemented using separate computers or using a single computer, or may be integrated with other computing functions of the system. Additionally or alternatively, at least some of the positioning and image processing functions may be performed using dedicated hardware.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention. In that regard, the drawings are not necessarily to scale. Accordingly, the foregoing description should not be read as pertaining only to the precise structures described and illustrated in the accompanying drawings, but rather should be read consistent with and as support to the following claims which are to have their fullest and fair scope.

What is claimed is:

1. A renal ablation and visualization system, comprising:
at least one input interface for 3-D image data of a renal region with at least a first anatomical structure;
a position sub-system generating 3-D mapping data of the renal region with at least a second anatomical structure;
an image processor structured and arranged to process the 3-D image data and the 3-D mapping data and generate a composite image of the renal region;
a display; and
a catheter with at least one ablation electrode,
wherein the composite image includes visualization of at least the first and second anatomical structures, and the at least one ablation electrode, the composite image includes dynamic visualization of movement of the at least one ablation electrode.

2. The system of claim 1, wherein the at least first anatomical structure comprises one from the group consisting of a renal vein, a lymph node, and soft tissue internal organ.

3. The system of claim 1, wherein the 3-D image data includes at least one from the group consisting of CT scans, MRI scans, 3-D ultrasonic images.

4. The system of claim 1, wherein the 3-D mapping data is acquired via magnetic sensing employing magnetic fields and responsive magnetic sensors.

5. The system of claim 1, wherein the system includes a user input interface structured and arranged to receive user input to tag at least one anatomical structure for relative enhanced visualization in the composite image.

6. The system of claim 1, wherein the system includes a user input interface structured and arranged to receive user input to tag at least one anatomical structure for relative diminished visualization in the composite image.

7. The system of claim 1, wherein the system includes user input interface structured and arranged to receiver user input to tag at least one anatomical structure for use by the image processor in processing at least one of the group consisting of the 3-D mapping data and the 3-D image data.

8. A renal ablation and visualization system, comprising:
at least one input interface for 3-D image data of a renal region with at least a first anatomical structure;
a position sub-system generating 3-D mapping data of the renal region with at least a second anatomical structure;
an image processor structured and arranged to process the 3-D image data and the 3-D mapping data and generate a composite image of the renal region, the image processor comprising:
a segmentation module structured and arranged to segment the 3-D image data to extract a 3-D surface profile;
a registration module structured and arranged to correlate the 3-D mapping data and the 3-D image data representing the 3-D surface profile by surfacing matching of the 3-D surface profile from the 3-D image data to a 3-D surface profile from the 3-D mapping data; and
a visualization module structured and arranged to generate the composite image using the correlated 3-D mapping data and at least the 3-D image data representing the 3-D surface profile; a display structured and arranged to display the composite image; and
a catheter with at least one ablation electrode,
wherein the composite image includes visualization of at least the first and second anatomical structures, and the at least one ablation electrode, the composite image includes dynamic visualization of movement of the at least one ablation electrode.

9. The system of claim 8, wherein the 3-D image data comprises at least one from the group consisting of CT scans, MRI scans, 3-D ultrasonic image data.

10. A renal ablation and visualization system, comprising:
a first input interface for 3-D image data of a renal region with at least a first anatomical structure;
a fluoroscopic imaging device providing 2-D image data of the renal region with at least a second anatomical structure, the 2-D image data having been acquired in a selected direction;
an image processor structured and arranged to process the 3-D image data and the 2-D image data and generate a composite image of the renal region, the image processor including a 3D/2D converter structured and arranged to reconstruct the 3-D image data in 3-D space and to compress the 3-D image data in the selected direction;
a display; and
a catheter with at least one ablation electrode,
wherein the composite image includes visualization of at least the first and second anatomical structures, and the at least one ablation electrode.

11. The system of claim 10, further comprising a user input interface structured and arranged to receive user tagging selection of an anatomical feature.

12. The system of claim 10, wherein the system includes a user input interface structured and arranged to receive user input to tag at least one anatomical structure for enhanced visualization in the composite image.

13. The system of claim 10, wherein the system includes user input interface structured and arranged to receiver user input to tag at least one anatomical structure for use by the image processor in processing at least one of the group consisting of the 2-D image data and the 3-D image data.

14. A renal ablation and visualization system, comprising:
at least one input interface for 3-D image data of a renal region with at least a renal vein;
a position sub-system generating 3-D mapping data of the renal region with at least a renal artery;
an image processor structured and arranged to process the 3-D image data and the 3-D mapping data and generate a composite image of the renal region, the image processor comprising:
a segmentation module structured and arranged to segment the 3-D image data to extract a 3-D surface profile;
a registration module structured and arranged to correlate the 3-D mapping data and the 3-D image data representing the 3-D surface profile by surfacing matching of the 3-D surface profile from the 3-D image data to a 3-D surface profile from the 3-D mapping data; and
a visualization module structured and arranged to generate the composite image using the correlated 3-D mapping data and at least the 3-D image data representing the 3-D surface profile;
a display structured and arranged to display the composite image;
a catheter with at least one ablation electrode,
wherein the composite image includes visualization of at least the renal vein, the renal artery and the at least one ablation electrode, the composite image includes dynamic visualization of movement of the at least one ablation electrode.

15. The system of claim 14, wherein the system includes user input interface structured and arranged to receive user input to tag at least one anatomical structure for enhanced visualization in the composite image.

16. The system of claim 14, wherein the system includes user input interface structured and arranged to receiver user input to tag at least one anatomical structure for use by the image processor in processing at least one of the group consisting of the 3-D mapping data and the 3-D image data.

17. A method of ablation a region of a renal artery, comprising:
providing a catheter with at least one electrode structured and arranged for ablation;
providing visualization of the renal artery and at least an adjacent anatomical structure comprising one from the group consisting of a renal vein, a lymph node, and an organ; and
selection a target site for ablation by the at least one electrode based on proximity of the adjacent anatomical structure.

18. The method of claim 17, wherein providing visualization includes providing visualization of the at least one electrode.

19. The method of claim 17, wherein providing visualization includes providing a composite image using a first image data and a second image data.

20. The method of claim 17, wherein providing visualization includes providing a composite image using a 2-D image data and a 3-D image data.

21. The method of claim 17, wherein providing visualization includes providing a composite image using 3-D mapping data and a 3-D image data.

22. The method of claim 17, wherein providing visualization includes providing a first 2-D image data and a second 2-D image data.

23. The method of claim 17, further comprising receiving user input on tagging at least an anatomical structure.

24. The method of claim 17, further comprising restricting blood flow in an adjacent renal vein.

25. The method of claim 17, further comprising preventing heat loss at the target site due to blood flow in an adjacent renal vein.
